(19) United States
(12) Patent Application Publication
(10) Pub. No.: US 2011/0098295 A1
(43) Pub. Date: Apr. 28, 2011

(54) METHODS OF TREATING ANXIETY, ITCHING AND PSYCHIATRIC DISORDERS

(76) Inventor: Min Zhuo, Toronto (CA)

(21) Appl. No.: 12/910,362

(22) Filed: Oct. 22, 2010

Related U.S. Application Data
(60) Provisional application No. 61/272,695, filed on Oct. 22, 2009.

(51) Int. Cl. A61K 31/5377; A61K 31/52; C07D 473/34; A61P 25/22; A61P 25/00; A61P 3/10; A61P 25/36

(52) U.S. Cl. 514/234.2; 514/263.38; 544/276; 544/118

(57) ABSTRACT

A method of treating an undesirable AC8-related condition in a mammal is provided comprising the step of inhibiting AC8 in the mammal

### CROSS-REFERENCE TO RELATED APPLICATION

This application claims the benefit of U.S. Provisional Patent Application No. 61/272,695, filed Oct. 22, 2009, and incorporates such provisional patent application in its entirety by reference.

### FIELD OF THE INVENTION

This invention generally relates to method of treating undesirable conditions associated with adenylyl cyclase subtype 8, including cognitive disorders such as anxiety and anxiety-related disorders, psychiatric disorders and excessive itching.

### BACKGROUND

The amygdala is known to play a critical role in anxiety. Although much is to be learned about anxiety at the synaptic level, anxiety is generally considered to be a concern or apprehension about what might happen. In both humans and animals, electrical stimulation of the amygdala elicits anxiety. A balance between excitatory and inhibitory transmission is critical for the mediation of anxiety-like behaviors. For example, hyperexcitation due to enhanced excitatory transmission or reduced inhibitory transmission can promote anxiety. The most widely prescribed classes of anxiolytic drugs, the benzodiazepines and the selective serotonin reuptake inhibitors, modulate GABA- and serotonin-mediated neurotransmission to reduce the neuronal excitability. Therefore, it is generally believed that the hyperexcitation of neural circuits including the amygdala, septo-hippocampal area, and prefrontal cortex, are mainly responsible for the anxiety disorders.

Acute or physiological anxiety is a key higher brain function and thought to be a means for controlling an animal's response to threatening or potentially threatening stimuli. However, excessive levels of anxiety or prolonged anxiety, or pathological anxiety, cause distress and suffering. Most of current medicinal treatments for anxiety do not act selectively on acute versus chronic anxiety. This is because most drugs act by modulating central excitatory and/or inhibitory transmission. Fewer drugs have been designed to selectively target proteins that are primarily involved in chronic anxiety.

It would be desirable, thus, to identify protein(s) directly involved in chronic anxiety and to develop treatments selectively targeting such protein(s).

### SUMMARY OF THE INVENTION

The effect of AC8 inhibition has now been elucidated. The present invention, thus, relates to the treatment of disorders from which inhibition of adenylyl cyclase subtype 8 (AC8) will benefit.

Accordingly, in one aspect, the invention provides a method of treating an undesirable AC8-related condition in a mammal in need of such treatment and comprises the step of inhibiting AC8 in the mammal.

In another aspect of the invention, a family of AC-inhibiting compounds is provided having the general formula (1):

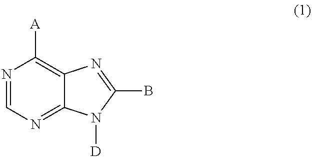

Another aspect of the present invention is directed to the novel AC-inhibiting compounds particularly 6-amino-9-(2-p-tolyloxy-ethyl)-9H-purine-8-thiol and 4-(9H-purin-6-yl)morpholine (also known as 6-morpholin-4-yl-7H-purine).

In a further aspect of the invention, a method of treating an undesirable AC8-related condition in a mammal is provided comprising administering to the mammal an AC8 inhibitor. In one embodiment the AC8 inhibitor is a compound having the general formula (1) as shown above.

In a further aspect, a pharmaceutical composition is provided comprising an AC8 inhibitor of the general formula (1) in combination with a pharmaceutically acceptable adjuvant.

These and other aspects of the invention will be described by reference to the detailed description and drawings.

### BRIEF DESCRIPTION OF THE DRAWINGS

FIG. 1 shows genetic evidence for the involvement of AC8 but not AC1 in behavioral anxiety. Wild-type, AC1 and AC8 KO mice were tested in the elevated plus maze (EPM) test. The EPM test is frequently used to measure anxiety levels in rodents and to screen potential anxiolytic drugs. Compared with wild-type and AC1 KO mice, AC8 KO mice spent significantly more time in the open arms, suggesting the reduced anxiety in mice lacking AC8. Data are presented as mean±SEM.*P<0.01 as compared with wild-type or AC1 knockout mice.

FIG. 2 shows AC8 KO mice subject of behavioral anxiety tests displayed decreased anxiety-like behavior. Mice were tested on the elevated plus maze every hour for three hours. A) The percentage of time spent by AC8 KO mice (n=6) in the open arms was greater than wild-type mice (n=3). B) The normalized data showing decreased anxiety-like behavior in AC8 KO mice.

FIG. 3 shows effects of AC8 inhibitor, Compound A, in an animal in the open field test, an animal model of behavioral anxiety. Increased open field activity in wild-type mice injected with AC8 inhibitor, Compound A (5 mg/kg, i.p.). Animals were placed in the open field immediately after an IP injection. A) Mice injected with AC8 inhibitor (filled circles; 5 mg/kg, i.p.; n=6) had significantly more ambulatory counts than mice injected with saline (open circles; n=5). *P<0.05. B) Mice injected with AC8 inhibitor (filled circles) traveled more than control mice injected with saline (open circles). *P<0.05

FIG. 4 shows reduced behavioral itching responses in AC8 KO mice. The scratching responses induced by intradermal injection of compound 48/80 (100 μg/50 μl) is significantly reduced in AC8 KO mice (n=9 mice) as compared with wild-type mice (n=7). *P<0.05. Data are presented as mean±SEM.

FIG. 5 shows reduced behavioral itching responses by i.p. injection of AC8 inhibitor, Compound A (5 mg/kg). The total scratching responses induced by intradermal injection of compound 48/80 (100 μg/50 μl) was found to be significantly reduced in mice receiving the injection Compound A (n=6 mice) as compared with wild-type mice (n=7). *P<0.05. Data are presented as mean±SEM.

### DETAILED DESCRIPTION OF THE INVENTION

In one aspect, a method of treating an undesirable AC8-related condition in a mammal is provided comprising the step of inhibiting AC8 in the mammal.

The term “undesirable AC8-related condition” is used herein to an adverse condition or disorder, that may or may not be a pathological condition, resulting from the activity or over-activity of AC8. Such conditions include, but are not limited to, cognitive disorders such as anxiety, stress-induced anxiety, pain-induced anxiety, generalized anxiety disorder (GAD), obsessive-compulsive disorder (OCD), panic disorder, post-traumatic stress disorder (PTSD), social phobia or social anxiety disorder; itching; type-2 diabetes; and morphine tolerance.

The term “mammal” is used herein to refer to human and non-human mammals, including but not limited to domestic animals (cats, dogs), livestock and wild animals.

In accordance with the method of treating an undesirable AC8-related condition in a mammal, AC8 is inhibited in the mammal. As one of skill in the art will appreciate, AC8 may be inhibited in a mammal at the nucleic acid level or at the protein level.

At the nucleic acid level, AC8 may be inhibited using techniques established in the art, for example, using anti-sense, snp or siRNA oligonucleotide technologies. AC-encoding nucleic acid molecules may be used to prepare oligonucleotides that are therapeutically useful to inhibit AC8. In this regard, oligonucleotides having therapeutic utility in the present method may be modified to include modified phosphorous or oxygen heteroatoms in the phosphate backbone, or short chain alkyl or cycloalkyl intersugar linages or short chain heteroatomic or heterocyclic intersugar linkages, such as phosphorothioates, phosphotriesters, methyl phosphonates, and phosphorodithioates. The therapeutic oligonucleotides may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides. Such therapeutic oligonucleotides may be introduced into tissues or cells to inhibit AC8 expression using techniques in the art including vectors (retroviral vectors, adenoviral vectors and DNA virus vectors) or physical techniques such as microinjection. The oligonucleotides may be directly administered in vivo or may be used to transfect cells in vitro which are then administered in vivo.

Immunological techniques may also be utilized to inhibit AC8, including the use of an AC8 antibody. Conventional methods may be used to prepare AC8 antibodies including polyclonal antisera or monoclonal antibodies, e.g. using established hybridoma technology.

At the protein level, synthetic AC8 inhibitors may be prepared, using standard chemical synthesis techniques, and utilized in the present methods of treatment. In one embodiment, AC8 inhibitors having the following general formula (1), and pharmaceutically acceptable salts, are provided:

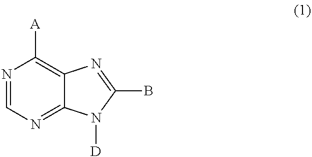

wherein A is selected from the group consisting of —NH₂, —NO₂, —NHR¹, —NR¹R², or a C₃-C₆ aromatic or non-aromatic ring structure or heterocyclic ring structure incorporating at least one heteroatom selected from N, O or S, said ring structure being optionally substituted with OH, halogen, NH₂, C₁-C₆ alkyl, C₁-C₆ alkanol or C₁-C₆ alkoxy, wherein R¹ and R² are independently selected from the group consisting of a C₁-C₆ alkyl, C₁-C₆ alkanol, C₁-C₆ alkoxy and C₁-C₆ carboxyalkyl;

B is selected from the group consisting of —H, —OH, —SH, —OR¹, —NH₂, —NO₂, —NHR¹, —NR¹R², halogen or —C₁-C₆ saturated or unsaturated alkyl group optionally substituted with one or more substituents selected from hydroxy, halogen, thio, OR¹, NH₂, NO₂, NHR¹, NR¹R², SR¹, wherein R¹ and R² are as defined above; and

D is selected from the group consisting of H, or C₁-C₆ alkyl or C₁-C₆ alkoxy, optionally substituted with NH₂, NHR¹, NR¹R², SH, SR¹, an unsubstituted C₃-C₇ cycloalkyl, phenyl or C₄-C₆ heterocyclic ring, or a substituted C₃-C₇ cycloalkyl, phenyl or C₄-C₆ heterocyclic ring having one or more substituents selected from the group consisting of C₁-C₆ alkyl, C₁-C₆ alkoxy, C₁-C₆ alkanoyl, C₁-C₆ carboxyalkyl, halogen or OH, wherein R¹ and R² are as defined above.

In embodiments of the invention, the AC-inhibiting compound is a compound of formula (1) in which A is NH₂, B is SH and D is C₁-C₆ alkoxy substituted with a phenyl group including a C₁-C₆ alkyl substituent, e.g. 6-amino-9-(2-p-tolyloxy-ethyl)-9H-purine-8-thiol; or a compound of formula (1) in which A is C₃-C₆ non-aromatic heterocyclic ring structure incorporating nitrogen and oxygen heteroatoms, and B and D are each hydrogen, e.g. 4-(9H-purin-6-yl)morpholine (also known as 6-morpholin-4-yl-7H-purine).

In another embodiment, the AC8 inhibitor, 6-amino-9-(2-p-tolyloxy-ethyl)-9H-purine-8-thiol is provided, herein referred to as Compound A, the chemical structure of which is as follows:

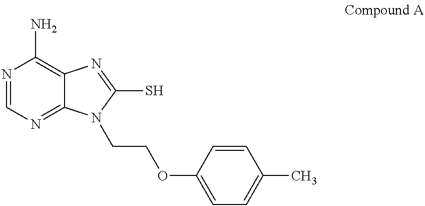

In another embodiment, the AC8 inhibitor, 4-(9H-purin-6-yl)morpholine (also known as 6-morpholin-4-yl-7H-purine), referred to herein as Compound B, is provided which has the following chemical structure:

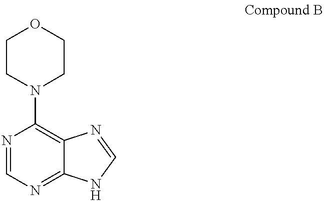

Encompassed within this invention, and falling within the definitions of Compound A and Compound B, are pharmaceutically acceptable salts of the compounds having the illustrated chemical formulae.

Inhibition of AC8 is useful to treat a mammal suffering from “generalized anxiety disorder”, GAD, which is an anxiety disorder characterized by chronic anxiety, exaggerated worry and tension, even when there is little or nothing to provoke such symptoms. People with generalized anxiety disorder can't seem to shake their concerns. Their worries are accompanied by physical symptoms, especially fatigue, headaches, muscle tension, muscle aches, difficulty swallowing, trembling, twitching, irritability, sweating, and hot flashes.

Inhibition of AC8 is useful to treat a mammal suffering from “obsessive-compulsive disorder”, OCD, which is an anxiety disorder characterized by recurrent, unwanted thoughts (obsessions) and/or repetitive behaviors (compulsions). Repetitive behaviors such as handwashing, counting, checking, or cleaning are often performed with the hope of preventing obsessive thoughts or making them go away. Performing these so-called “rituals,” however, provides only temporary relief, and not performing them markedly increases anxiety. People with OCD may be plagued by persistent, unwelcome thoughts or images, or by the urgent need to engage in certain rituals. They may be obsessed with germs or dirt, and repeatedly wash their hands. Such people may be filled with doubt and frequently feel the need to check things repeatedly.

Inhibition of AC8 is useful to treat a mammal suffering from “panic disorder”, which is an anxiety disorder characterized by unexpected and repeated episodes of intense fear accompanied by physical symptoms that may include chest pain, heart palpitations, shortness of breath, dizziness, or abdominal distress. People with panic disorder have feelings of terror that strike suddenly and repeatedly with no warning. During a panic attack, a person's heart will often pound and the person may feel sweaty, weak, faint, or dizzy. Hands may tingle or feel numb, and the person can feel flushed or chilled. There may be nausea, chest pain or smothering sensations, a sense of unreality, or fear of impending doom or loss of control.

Inhibition of AC8 is useful to treat a mammal suffering from “post-traumatic stress disorder”, PTSD, which is an anxiety disorder that can develop after exposure to a terrifying event or ordeal in which grave physical harm occurred or was threatened. Traumatic events that may trigger PTSD include violent personal assaults, natural or human-caused disasters, accidents, or military combat. People with PTSD have persistent frightening thoughts and memories of their ordeal, and often feel emotionally numb, especially towards people with whom they were once close. Such people often experience sleep problems, feel detached or numb, and can be easily startled.

Inhibition of AC8 is useful to treat a mammal suffering from “social phobia”, or “social anxiety disorder”, which is an anxiety disorder characterized by overwhelming anxiety and excessive self-consciousness in everyday social situations. Social phobia may include fear of speaking in formal or informal situations, or eating or drinking in front of others or, in its most severe form, may be so broad that a person experiences symptoms almost anytime they are around other people. People with social phobia have a persistent, intense, and chronic fear of being watched and judged by others and being embarrassed or humiliated by their own actions. Their fear may be so severe that it interferes with work or school, and other ordinary activities. Physical symptoms often accompany the intense anxiety of social phobia and include blushing, profuse sweating, trembling, nausea, and difficulty talking

Thus, administration of a compound that inhibits AC8 activity in accordance with the invention is effective to treat anxiety disorders such as GAD, OCD, panic disorder, PTSD and social anxiety disorder.

Inhibition of AC8 is useful to treat a mammal diagnosed with type-2 diabetes. Type 2 diabetes, the most common form of diabetes, usually appears in adults, often in middle age. Type 2 diabetes is often linked with obesity and its onset can sometimes be delayed or the disease controlled with diet and exercise. Obesity and physical inactivity are two risk factors for type 2 diabetes. In a mild form, it can go undetected for many years. Untreated diabetes can lead to other serious medical problems, including cardiovascular disease. In an animal model of diabetes, AC8 was found to be significantly increased in both diabetic β and α-cells (Guenifi et al., 2000). Thus, inhibition of AC8 to reduce higher AC8 activity in these cells in type-2 diabetes, and normalize the release of insulin is useful in a treatment according to the invention. In this regard, administration of a compound that inhibits AC8 activity in the islets is effective to treat type-2 diabetes in a mammal.

Inhibition of AC8 is useful to treat a mammal suffering from itching. Itching is an uncomfortable sensation in the skin that feels as if something is crawling on the skin or in the skin, and itching generally creates an urge in the person to scratch the affected area. Itch often accompanies many conditions. Probably the most common cause of itch is psychological, and results from stress, anxiety, etc. Stress also can aggravate an itch from other causes. Dry skin is another frequent cause of itch. Other causes include metabolic and endocrine disorders (e.g., liver or kidney disease, hyperthyroidism), cancers (e.g., lymphoma), reactions to drugs, and diseases of the blood (e.g., polycythemia vera). Infections and infestations of the skin are another cause of itch. Common infectious causes of itch include a fungal infection of the crotch (tinea cruris) commonly known as jock itch as well as vaginal itching and/or anal itching from sexually-transmitted diseases (STDs) or other types of infections. Another type of parasitic infection resulting in itch is the so-called swimmer's itch. Swimmer's itch, also called cercarial dermatitis, is a skin rash caused by an allergic reaction to infection with certain parasites of birds and mammals that are released from infected snails who swim in fresh and salt water. Itch may also result from skin infestation by body lice, including pubic lice. As described in more detail herein, AC8 inhibition resulted in reduced itching responses to histamine injection. Thus, administration of a compound that inhibits AC8 activity in accordance with the invention is effective to treat itching in a mammal.

Inhibition of AC8 is useful to enhance morphine analgesia and/or reduce morphine tolerance in a mammal undergoing treatment with morphine. Morphine tolerance (loss of morphine analgesic effects) can occur with patients who use morphine for an extended period of time. In both humans and animals, repeated usage of morphine over several days or a few weeks will lead to diminished analgesic effects. Among patients with chronic pain such as neuropathic pain, cancer pain, pain-medicines can be needed for very long periods of time. Additionally, a side effect of morphine treatment can be itching. Thus, AC8 inhibitors are especially beneficial in this context.

AC8 inhibitors in accordance with the invention, having the general formula (1), may be administered alone or as a pharmaceutical composition comprising the inhibitor and at least one pharmaceutically acceptable adjuvant to treat an undesirable AC8-related condition. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally in the art such as diluents, excipients and the like. Reference may be made to “Remington's: The Science and Practice of Pharmacy”, 21st Ed., Lippincott Williams & Wilkins, 2005, for guidance on drug formulations generally. The selection of adjuvant will depend on the nature of the inhibitor and the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations, for example, for nasal delivery, may also be prepared in which suitable propellant adjuvants are used. The inhibitor will like-wise be combined with adjuvants suitable for rectal, permucous or percutaneous administration. Other adjuvants may also be added to the composition regardless of how it is to be administered, for example, anti-microbial agents may be added to the composition to prevent microbial growth over prolonged storage periods.

The pharmaceutical compositions of the invention may include one or more pharmaceutically acceptable salts. A “pharmaceutically acceptable salt” refers to a salt that retains the desired biological activity of the parent compound and does not impart any undesired toxicological effects (see e.g., Berge, S. M. et al, (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In accordance with the invention, a therapeutically effective amount of an AC8 inhibitor is administered to a mammal in the treatment of an undesirable AC8-related condition. The term “therapeutically effective amount” is an amount of the inhibitor indicated for treatment of the condition while not exceeding an amount which may cause significant adverse effects. As one of skill in the art will appreciate, the appropriate dosage of AC8 for treating an AC8-related condition may vary within wide ranges (e.g. about 0.5 mg to 1000 mg) according to the therapeutic indication and the route of administration, and also to the age and weight of the patient.

In one embodiment, an AC8 inhibitor is administered to the hippocampus, amygdala and/or cortex of a mammal in need of treatment for an AC8-related condition. In this regard, the AC8 inhibitor, such as a compound of general formula (1) is prepared in an appropriate manner for such administration using techniques well-established in the art.

Embodiments of the present invention are described by reference to the following specific examples which are not to be construed as limiting.

### EXAMPLES

### Gene Knockout Mice

Adult AC8 knockout (KO) mice were used in which AC8 has been reduced by homologous recombination in embryonic stem cells (see Schafer et al., 2000). AC8 KO mice demonstrate a compromise in calcium-stimulated AC activity in the hippocampus, hypothalamus, thalamus, and brainstem.

Control wild-type (WT) mice were littermates of the KO mice. Mice were housed on a 12 h:12 h light:dark cycle with ad libitum access to rodent chow. All mouse protocols used were approved by The Animal Care and Use Committee at the University of Toronto.

### Animal Models of Behavioral Anxiety

### Elevated Plus Maze (EPM)

The elevated plus maze (Med Associates, St. Albans, Vt.) consisted of two open arms and two closed arms situated opposite each other. The maze was situated approximately 70 cm from the floor. For each test, mice were individually placed in the center square and allowed to move freely for 5 minutes. The number of entries and time spent in each arm were recorded. The animals were given i.p. injection of Compound A or saline 30 minutes before testing. A video camera tracking system (Ethovision, Noldus, Va.) was used to generate the traces.

### Open Field Safety Test

A round, green, plastic tub with diameter 43.5 cm was used to create an open arena for the mouse to travel. The floor was made of clear Plexiglas. Each trial consisted of exposing the mouse to a bright light (685 lux) in the arena for the first minute, followed immediately by exposing the mouse only to dim light (1 lux) for another minute. The mouse's movements were captured using video camera tracking software (Ethovision, Noldus Va.) and the distance from the border of the arena, the distance from the centre of the arena, and the total distance traveled were measured. The percent difference in these factors between bright and dim light periods were also calculated. After each 2 min testing session, the mouse was removed and placed back in its home cage and the arena was cleaned with 70% ethanol solution.

### Behavioral Itching Test

Mutant KO mice and littermate wild-type mice were used in the behaviour experiments. Mice were shaved at the back of the neck where intradermal injections of compound 48/80, which releases histamine from the mast cells, were then given. Hindlimb scratching behaviour directed towards the shaved area at the back of the neck was observed for 30 min at 5-min intervals.

### Biochemical Screening Test for Novel Inhibitors for AC8

For AC8 expression vector pcDNA3-AC1 transfection, HEK293 cells were plated onto 60-mm-diameter dishes at a density of 1×10⁶ per plate, grown overnight and transfected with pcDNA3-AC1 (0.8 μg DNA per plate) by Lipofectamine 2000 (Invitrogen). Stable transfected clones were selected in culture media containing 0.8 mg/ml G418 (Invitrogen, CA) and maintained in this media. For transient expression of other AC isoforms in HEK293 cells, HEK293 cells were plated in 96 well tissue culture dishes and transfected with plasmids for AC5, respectively, experiments were carried out at 48 h after transfection.

The HEK293 cells expressing ACs were harvested and lysed in 0.1 M HCl after different treatments. Direct cAMP measurements were performed using the direct cAMP enzyme immunoassay kit (Assay Designs, MI) and optical density values were measured at 405 nm by the microplate reader. Phosphodiesterase was inhibited by the addition of 1 mM 3-isobutyl-1 methylxanthine (Sigma, Mo.) to cultures.

### CRE Luciferase Reporter Assay

The HEK293 cells were subcultured into 96-well plates in the absence of antibiotics and grown overnight and transfected with the pGL3-CRE-firefly luciferase and pGL3-CMV-Renilla luciferase constructs (0.25 μg DNA per well) by Lipofectamine 2000 reagent. The transfected cells were incubated overnight, and media were changed to DMEM containing 10% fetal bovine serum. After 48 h, the cells were treated with 10 μM forskolin, 10 μM A23187 and 2 mM CaCl₂, or a combination of 10 μM forskolin, 10 μM A23187 and 2 mM CaCl₂, in the absence or presence of each chemical tested at the concentration of 100 μM. At the end of 6 hours, incubation cells were harvested and luciferase activity was assayed by Dual-Luciferase Reporter Assay System (Promega). Relative light units were measured using a SIRIUS luminometer.

### Whole-Cell Patch Clamp Recordings

Coronal brain slices (300 μm) at the level of the ACC were prepared using standard methods (Wu et al., 2005). Slices were transferred to submerged recovery chamber with oxygenated (95% O₂ and 5% CO₂) artificial cerebrospinal fluid (ACSF) containing (in mM: 124 NaCl, 2.5 KCl, 2 CaCl₂, 1 MgSO₄, 25 NaHCO₃, 1 NaH₂PO₄, 10 glucose) at room temperature for at least 1 hr. Experiments were performed in a recording chamber on the stage of a BX51W1 microscope equipped with infrared DIC optics for visualization. Excitatory postsynaptic currents (EPSCs) were recorded from layer II/III neurons with an Axon 200B amplifier (Axon Instruments, CA) and the stimulations were delivered by a bipolar tungsten stimulating electrode placed in layer V of the ACC. Alpha-amino-3-hydroxy-5-methylisoxazole-4-propionic acid (AMPA) receptor-mediated EPSCs were induced by repetitive stimulations at 0.05 Hz and neurons were voltage clamped at −70 mV in the presence of AP5 (50 μM). The recording pipettes (3-5 MΩ were filled with a solution containing (in mM: 145 K-gluconate, 5 NaCl, 1 MgCl₂, 0.2 EGTA, 10 HEPES, 2 Mg-ATP, 0.1 Na₃-GTP and 10 phosphocreatine disodium (adjusted to pH 7.2 with KOH). The internal solution (in mM) 140 cesium methanesulfonate, 5 NaCl, 0.5 EGTA, 10 HEPES, 2 MgATP, 0.1 Na₃GTP, 0.1 spermine, 2 QX-314 bromide and 10 phosphocreatine disodium (adjusted to pH 7.2 with CsOH) was used in the rectification of AMPA receptor-mediated transmission experiment. For miniature EPSC (mEPSC) recording, 0.5 μM TTX was added in the perfusion solution. Picrotoxin (100 μM) was always present to block GABA_{A} receptor-mediated inhibitory synaptic currents in all experiments. Access resistance was 15-30 MΩ and monitored throughout the experiment. Data were discarded if access resistance changed more than 15% during an experiment. Data were filtered at 1 kHz, and digitized at 10 kHz.

### Results

### Genetic Evidence for AC8 in Behavioral Anxiety

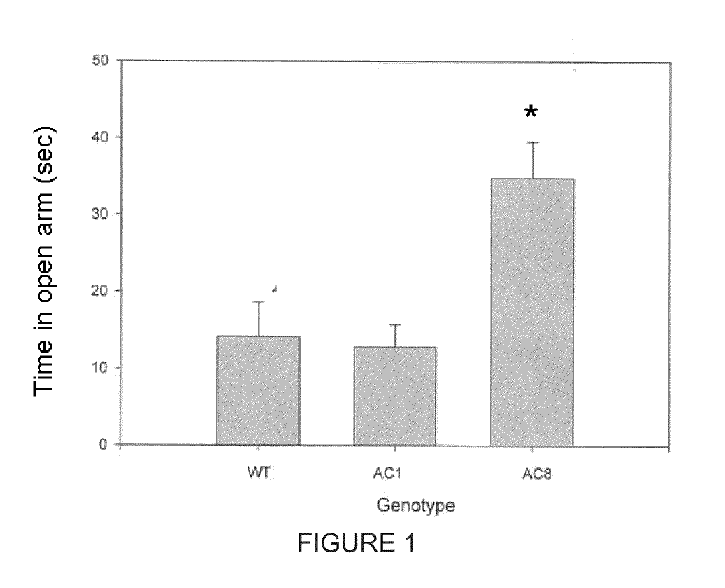

To determine the potential contribution of AC8 to behavioral anxiety, wild-type and AC8 knockout mice were examined in the elevated plus maze test. It was found that there is a significant difference in the percent time spent in the open arms, with AC8 KO mice spending the greatest percent time in the open arms (P<0.01, one way ANOVA, AC8 KO n=6, WT=6) See FIG. 1. There were also significant differences in the amount of time spent in the open zone farthest from the centre (P<0.01, one way ANOVA), as well as the number of entries into the farthest zone of the open arm (P<0.001, one way ANOVA). These results consistently suggest that AC8 gene deletion reduce behavioral anxiety in mice.

To examine if these effects may be AC subtype selective, the behavioral anxiety in mice lacking AC1, another isoform of calcium-stimulated adenylyl cyclase, was also measured. It was found that AC1 gene deletion did not significantly affect behavioral anxiety responses as compared with WT mice (AC8, n=8 mice) See FIG. 1. This finding indicates that the involvement of AC8 in behavioral anxiety is subtype-selective.

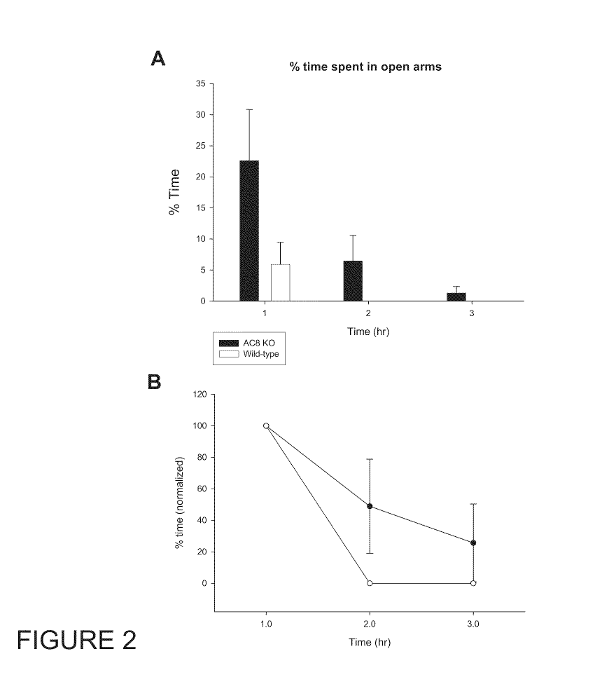

Repetitive measurements in the EPM lead to desensitization of responses. Normal mice tend to stay in the closed arms after repetitive measurement. See FIG. 2. AC8 KO mice, however, spent more time in open arms, suggesting that AC8 activity can be critical for repetitive behavioral anxiety responses.

The open field safety test was also performed to determine the response of the mice to a safety signal. First, a bright light was shown on an arena for 1 min, followed immediately by a dim light for one minute. The mice were allowed to roam the arena freely under both conditions. Although all mice responded to the safety signal as measured by the distance moved towards the centre of the arena, the AC8 KO mice (n=9) traveled more into the centre in the bright light (P<0.01, one way ANOVA). By contrast, the AC1 KO mice responded in a manner similar to WT mice (n=8). Thus, these results provide additional evidence that AC8 KO mice have a reduced behavioral anxiety.

### Screening for AC8 Inhibitors

A set of biochemical screening tests were carried out to search for potential selective AC8 inhibitors. cAMP assay, gene-activation (pCREB) assay in AC8 expressed cell lines, as well as integrative physiological experiments were employed to screen for potential AC8 inhibitors. The effect of Compound A and B on AC8 was determined. It was found that both Compound A and Compound B at 100 μM produced significant inhibition of AC8 activity. See Table 1.

TABLE 1
Effects of Compound A and Compound B on the
activity of adenylyl cyclase subtype 8 and 5

Percent Inhibition

| | AC8 | AC5 |
|---|---|---|
| Compound A (100 μM) | 88 ± 2% inhibition | 25 ± 4% |
| Compound B (100 μM) | 86 ± 2 | |

### Behavioral Effects of AC8 Inhibitors on Anxiety

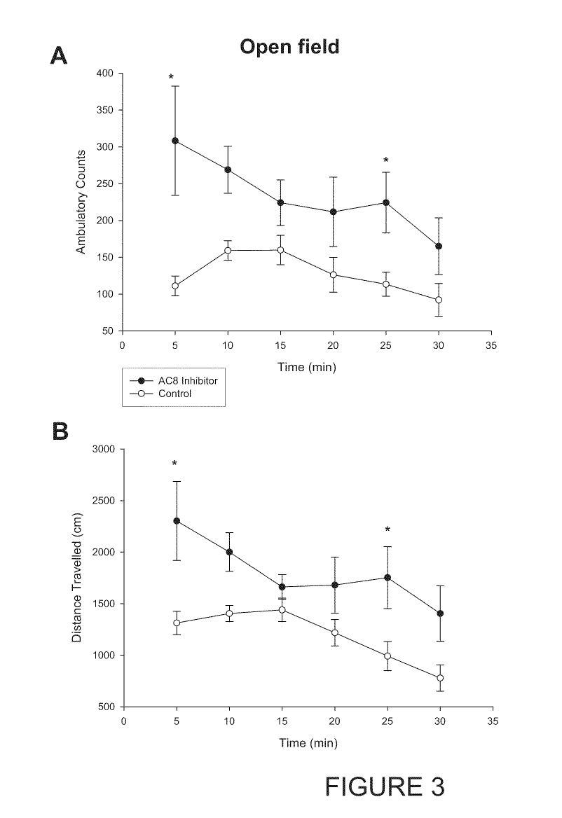

In behavioral studies in an animal model of anxiety (Wu et al. PLoS One. 2007 Jan. 24; 2 (1):e167; Xia et al. Mol. Brain. 2010 Aug. 2; 3:23), it was found that systemic injection of Compound A (10 mg/kg) produced significant anti-anxiety effects in the open field test (n=7 mice). See FIG. 3.

Given the foregoing results, inhibition of AC8 using AC8 inhibitors such as Compound A and Compound B is expected to be useful to treat type-2 diabetes, since AC8 is upregulated in diabetic GK rat islets (Guienifi et al., 2000; Portela-Gpmes and Abdel-Halim, 2002); to enhance morphine analgesic effects during chronic use of morphine since AC8 gene deletion affected morphine tolerance (Li et al., 2006); and to treat pain, and pain-related cognitive disorders since AC8 is located in central nuclei such as the anterior cingulate cortex, insular cortex that involved in pain perception (Xia et al., 1991; Zhuo, 2007).

### Behavioral Effects of AC8 Inhibitors on Itching

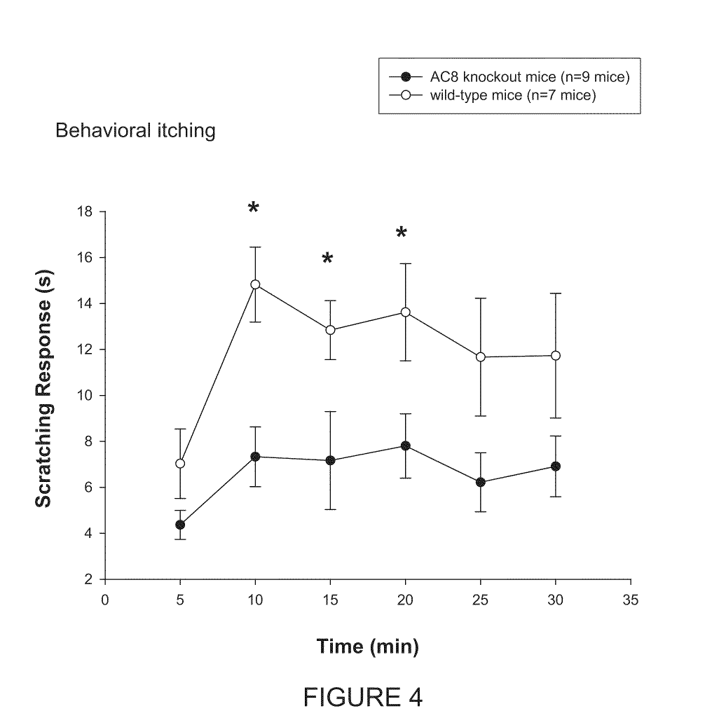

The effects of genetic deletion of AC8 on animal behavioral responses to itch stimulus was examined. It was found that the scratching responses in AC8 KO mice were significantly reduced as compared with that of wild-type mice. See FIG. 4.

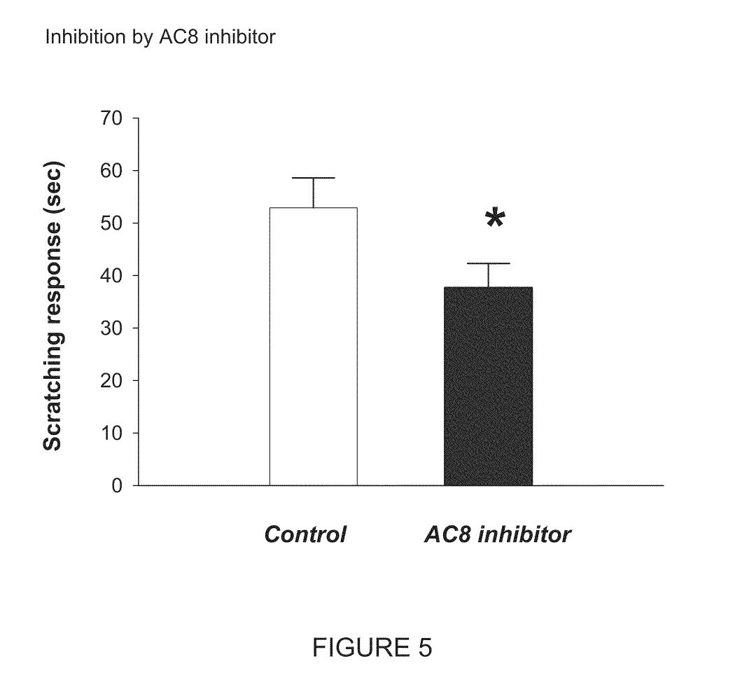

In behavioral studies in an animal model of itch (Sun and Chen, Nature. 2007; 448(7154):700-3. Epub 2007 July 25), it was found that systemic injection of Compound A (5 mg/kg) produced significant anti-itching effects in the itching test. See FIG. 5.

### REFERENCES

Davis, M. (1992). The role of the amygdala in fear and anxiety Annu Rev Neurosci 15, 353-375.
Davis, M., Rainnie, D., and Cassell, M. (1994). Neurotransmission in the rat amygdala related to fear and anxiety. Trends Neurosci 17, 208-214.
Etkin, A., and Wager, T. D. (2007). Functional neuroimaging of anxiety: a meta-analysis of emotional processing in PTSD, social anxiety disorder, and specific phobia. Am J Psychiatry 164, 1476-1488.
Gorman, J. M. (2001). Generalized anxiety disorder. Clin Cornerstone 3, 37-46.
Guenifi, A., Portela-Gomes, G. M., Grimelius, L., Efendic, S., and Abdel-Halim, S. M. (2000). Adenylyl cyclase isoform expression in non-diabetic and diabetic Goto-Kakizaki (GK) rat pancreas. Evidence for distinct overexpression of type-8 adenylyl cyclase in diabetic GK rat islets. Histochem Cell Biol 113, 81-89.
LeDoux, J. E. (2000). Emotion circuits in the brain. Annu Rev Neurosci 23, 155-184.
Li, S., Lee, M. L., Bruchas, M. R., Chan, G. C., Storm, D. R., and Chavkin, C. (2006). Calmodulin-stimulated adenylyl cyclase gene deletion affects morphine responses. Mol Pharmacol 70, 1742-1749.
Portela-Gomes, G. M., and Abdel-Halim, S. M. (2002). Overexpression of Gs proteins and adenylyl cyclase in normal and diabetic islets. Pancreas 25, 176-181.
Schaefer, M. L., Wong, S. T., Wozniak, D. F., Muglia, L. M., Liauw, J. A., Zhuo, M., Nardi, A., Hartman, R. E., Vogt, S. K., Luedke, C. E., et al. (2000). Altered stress-induced anxiety in adenylyl cyclase type VIII-deficient mice. J Neurosci 20, 4809-4820.
Wu, L. J., Zhao, M. G., Toyoda, H., Ko, S. W., and Zhuo, M. (2005). Kainate receptor-mediated synaptic transmission in the adult anterior cingulate cortex. J Neurophysiol 94, 1805-1813.
Xia, Z. G., Refsdal, C. D., Merchant, K. M., Dorsa, D. M., and Storm, D. R. (1991). Distribution of mRNA for the calmodulin-sensitive adenylate cyclase in rat brain: expression in areas associated with learning and memory. Neuron 6, 431-443.

All documents referred to herein are incorporated herein by reference, and Applicant reserves the right to incorporate in part or in whole any such document.

We claim:

1. A method of treating an undesirable AC8-related condition in a mammal comprising the step of inhibiting AC8 in the mammal.

2. The method of claim 1, wherein the undesirable AC8-related condition is selected from the group consisting of cognitive disorders such as anxiety, stress-induced anxiety, pain-induced anxiety, generalized anxiety disorder (GAD), obsessive-compulsive disorder (OCD), panic disorder, post-traumatic stress disorder (PTSD), social phobia or social anxiety disorder; itching; type-2 diabetes; and morphine tolerance.

3. The method of claim 2, wherein the condition is a cognitive disorder.

4. The method of claim 3, wherein the condition is anxiety or an anxiety disorder.

5. The method of claim 2, wherein the condition is itching.

6. The method of claim 1, wherein AC8 is inhibited in the mammal by administration of an AC8 inhibitor.

7. The method of claim 6, wherein the AC8 inhibitor is a compound having the following general formula (1): 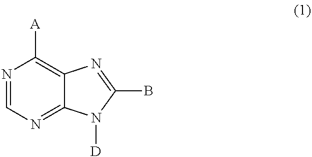
or a pharmaceutically acceptable salt thereof,
wherein A is selected from the group consisting of —NH₂, —NO₂, —NHR¹, —NR¹R², —SR¹, or a C₃-C₆ aromatic or non-aromatic ring structure or heterocyclic ring structure incorporating at least one heteroatom selected from N, O or S, said ring structure being optionally substituted with OH, halogen, NH₂, C₁-C₆ alkyl, C₁-C₆ alkanol or C₁-C₆ alkoxy, wherein R¹ and R² are independently selected from the group consisting of a C₁-C₆ alkyl, C₁-C₆ alkanol, C₁-C₆ alkoxy and C₁-C₆ carboxyalkyl;
B is selected from the group consisting of —H, —OH, —SH, —OR¹, —NH₂, —NO₂, —NHR¹, —NR¹R², —SR¹, halogen or —C₁-C₆ saturated or unsaturated alkyl group optionally substituted with one or more substituents selected from hydroxy, halogen, thio, OR¹, NH₂, NO₂, NHR¹, NR¹R², SR¹, wherein R¹ and R² are as defined above; and
D is selected from the group consisting of H, or C₁-C₆ alkyl or C₁-C₆ alkoxy, optionally substituted with NH₂, NHR¹, NR¹R², SH, SR¹, an unsubstituted C₃-C₇ cycloalkyl, phenyl or C₄-C₆ heterocyclic ring, or a substituted C₃-C₇ cycloalkyl, phenyl or C₄-C₆ heterocyclic ring having one or more substituents selected from the group consisting of C₁-C₆ alkyl, C₁-C₆ alkoxy, C₁-C₆ alkanoyl, C₁-C₆ carboxyalkyl, halogen or OH, wherein R¹ and R² are as defined above.

8. The method of claim 7, wherein the inhibitor is a compound of formula (1) in which A is NH₂, B is SH and D is C₁-C₆ alkoxy substituted with a phenyl group including a C₁-C₆ alkyl substituent.

9. The method of claim 8, wherein the compound is 6-amino-9-(2-p-tolyloxy-ethyl)-9H-purine-8-thiol.

10. The method of claim 7, wherein the inhibitor is a compound of formula (1) in which A is C₃-C₆ non-aromatic heterocyclic ring structure incorporating nitrogen and oxygen heteroatoms, and B and D are each hydrogen.

11. The method of claim 10, wherein the compound is 4-(9H-purin-6-yl)morpholine.

12. The method of claim 1, wherein the compound is administered to the hippocampus, amygdala and/or cortex of the patient.

13. A compound having the general formula (1): 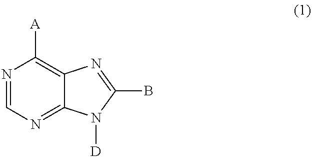
wherein A is selected from the group consisting of —NH₂, —NO₂, —NHR¹, —NR¹R², SR¹, or a C₃-C₆ aromatic or non-aromatic ring structure or heterocyclic ring structure incorporating at least one heteroatom selected from N, O or S, said ring structure being optionally substituted with OH, halogen, NH₂, C₁-C₆ alkyl, C₁-C₆ alkanol or C₁-C₆ alkoxy, wherein R¹ and R² are independently selected from the group consisting of a C₁-C₆ alkyl, C₁-C₆ alkanol, C₁-C₆ alkoxy and C₁-C₆ carboxyalkyl;
B is selected from the group consisting of —H, —OH, —SH, —OR¹, —NH₂, —NO₂, —NHR¹, —NR¹R², —SR¹, halogen or —C₁-C₆ saturated or unsaturated alkyl group optionally substituted with one or more substituents selected from hydroxy, halogen, thio, OR¹, NH₂, NO₂, NHR¹, NR¹R², SR¹, wherein R¹ and R² are as defined above; and
D is selected from the group consisting of H, or C₁-C₆ alkyl or C₁-C₆ alkoxy, optionally substituted with NH₂, NHR¹, NR¹R², SH, SR¹, an unsubstituted C₃-C₇ cycloalkyl, phenyl or C₄-C₆ heterocyclic ring, or a substituted C₃-C₇ cycloalkyl, phenyl or C₄-C₆ heterocyclic ring having one or more substituents selected from the group consisting of C₁-C₆ alkyl, C₁-C₆ alkoxy, C₁-C₆ alkanoyl, C₁-C₆ carboxyalkyl, halogen or OH, wherein R¹ and R² are as defined above.

14. The compound of claim 12, which is 6-amino-9-(2-p-tolyloxy-ethyl)-9H-purine-8-thiol or 4-(9H-purin-6-yl)morpholine.

15. An AC8-inhibiting pharmaceutical composition comprising the compound of claim 12 in combination with a pharmaceutically acceptable adjuvant.

16. The composition of claim 15, wherein the compound is 6-amino-9-(2-p-tolyloxy-ethyl)-9H-purine-8-thiol or a pharmaceutically acceptable salt thereof.

17. The composition of claim 15, wherein the compound is 4-(9H-purin-6-yl)morpholine or a pharmaceutically acceptable salt thereof.

18. A method of inhibiting AC8 comprising the step of exposing AC8 to a compound as defined in claim 13.

19. The method of claim 18, using the compound of claim 14.

20. The method of claim 18, using the compound of claim 16.
